(19) United States
(12) Patent Application Publication
(10) Pub. No.: US 2024/0374375 A1
(43) Pub. Date: Nov. 14, 2024

(54) Intraocular Lens with Power Factor and Structure for Improved Peripheral Vision

(71) Applicant: AMO Groningen B.V., Groningen (NL)

(72) Inventors: Dmitry Romashchenko, Groningen (NL); Carmen Canovas Vidal, Groningen (NL); Aixa Alarcon Heredia, Haren (NL); Marrie van der Mooren, Engelbert (NL); Mark David Jenkins Sanchez, Groningen (NL); Emmanuel Franck Gounou, Groningen (NL)

(21) Appl. No.: 18/623,370

(22) Filed: Apr. 1, 2024

Related U.S. Application Data
(60) Provisional application No. 63/493,565, filed on Mar. 31, 2023.

(51) Int. Cl. A61F 2/16

(52) U.S. Cl. CPC A61F 2/164; A61F 2002/1681

(57) ABSTRACT

Lenses and methods are provided for improving peripheral and central vision for patients. The lenses improve vision by having an optic configured to provide optimal peripheral vision that minimizes off-axis astigmatism at, for example, 20 degree eccentricity, while maximizing on-axis visual acuity and contrast sensitivity. The method provides for the design of an IOL that reduces optical errors in an image produced at a peripheral retinal location of a patient's eye disposed at a distance from the fovea, while maximizing on-axis visual acuity and contrast sensitivity.

### FIELD OF THE DISCLOSURE

Embodiments of the present disclosure relate to apparatuses, systems, and methods of manufacturing intraocular lenses that provide corrective vision with improvements to peripheral vision as well.

### BACKGROUND

Embodiments of the present disclosure relate to vision treatment techniques and in particular, to ophthalmic lenses such as intraocular lenses (IOLs) including, for example, phakic IOLs and piggyback IOLs (i.e. IOLs implanted in an eye already having an IOL).

Intraocular Lenses (IOLs) may be used for restoring visual performance after a cataract or other ophthalmic procedure in which the natural crystalline lens is replaced with or supplemented by implantation of an IOL. When such a procedure changes the optics of the eye, generally a goal is to improve vision in the central field. Recent studies have found that, when a monofocal IOL is implanted, peripheral aberrations are changed, and that these aberrations differ significantly from those of normal, phakic eyes. The predominant change is seen with respect to peripheral astigmatism, which is the main peripheral aberration in the natural eye, followed by sphere, and then higher order aberrations. Such changes may have an impact on overall functional vision, including the ability to drive, the risk of falling, postural stability and/or detection ability.

There are also certain retinal conditions that reduce central vision, such as age related macular degeneration (AMD) or a central scotoma. Other diseases may impact central vision, even at a very young age, such as Stargardt disease, Best disease, and inverse retinitis pigmentosa. The visual outcome for patients suffering from these conditions may be improved by improving peripheral vision.

Peripheral vision can also be degraded by Glaucoma. Glaucoma affects 2% of the population above the age of 40. Patients with glaucoma gradually lose peripheral vision as a result of damage to the optic nerve. Central vision may get degraded at very late stages of the disease. Significant disabilities in daily life can occur due to glaucoma, including problems with walking, balance, risk of falling and driving. Patients suffering from Glaucoma can benefit from IOLs that improve both central as well as peripheral vision.

In light of the above, lenses that improve peripheral vision are needed.

### BRIEF SUMMARY

The present invention solves the above problems by providing lenses having ranges of power shape factor and diopter label power that correspond to lenses having optimized on-axis visual acuity and contrast sensitivity and having a blur parameter of less than 1.8 diopters (D), which therefore improve peripheral vision. Furthermore, lenses are provided having shape factors and diopter label powers that fall within sub-ranges that correspond to an optimal peripheral vision lens that minimizes off-axis astigmatism at 20 degree eccentricity, while maximizing on-axis visual acuity and contrast sensitivity, wherein the optimization parameters are described herein.

In particular, a first embodiment of an intraocular lens comprises a lens body, wherein the lens body comprises: a refractive index between 1.40 and 1.50 inclusive, a diopter label power between 17 D and 23 D, inclusive, and a power shape factor that is less than or equal to −1.0 and greater than or equal to −2.5. Advantageously, providing an intraocular lens according to the label power range and power shape factor range of the first embodiment allows for correction of peripheral astigmatism in accordance with a blur parameter of less than 1.8 D, while optimizing on-axis visual acuity. Within these ranges, the power shape factor is stable, meaning that the structural features may be selected in order to produce lenses that are predictably capable of producing the effect of reducing off-axis astigmatism at 20 degree eccentricity while optimizing on-axis visual acuity. These ranges are critical for allowing predictable correction of peripheral astigmatism for patients having a dioptric power requirement that is less than a transition point power. The transition point power is the label power that corresponds to the shape factor of a plano-convex lens, which is −1.

The lens body may comprise a central thickness that is between 0.62 mm and 1.0 mm. The intraocular lens may further comprise an anterior haptic connected to the lens body, and the lens may comprise a vault height that is between 0.34 mm and 0.65 mm. The lens may comprise a first surface having an anterior surface curvature greater than −0.077 mm⁻¹ and less than 0.00 mm⁻¹ and a second surface having a posterior surface curvature greater than −0.355 mm⁻¹ and less than −0.130 mm⁻¹. Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort when inserted in a patient. The respective selection of these parameters further allows the lenses to have a power shape factor that is even more stable within the ranges of the invention, to further aid in the ability of the lenses to predictably improve peripheral vision.

The lens may comprise a diopter label power that is between 18 D and 22 D, inclusive. The power shape factor may be less than or equal to −1.1 and greater than or equal to −2.0. Advantageously, these ranges of label power and power shape factor result in a lens that is optimized such that the lens minimizes off-axis astigmatism while maximizing on-axis visual acuity, for lenses having a label power less than the transition point power. Across these ranges, the power shape factor of the lens is particularly stable which is critical for allowing for a greater ability to predict the structural features of the lens that result in optimization. The lens may comprise a central thickness that is between 0.70 mm and 0.90 mm. An anterior haptic may be connected to the lens body, and wherein the lens may comprise a vault height that is between 0.40 mm and 0.60 mm. The lens may comprise a first surface having an anterior surface curvature greater than −0.071 mm⁻¹ and less than −0.00 mm⁻¹ and a posterior surface curvature greater than −0.355 mm⁻¹ and less than −0.134 mm⁻¹. Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of optimization, to further aid in the ability of the lenses to predictably minimize peripheral vision while maximizing on-axis visual acuity.

The power shape factor may be calculated using paraxially defined curvature radii of the first surface and the second surface.

The first surface of the lens may comprise a concave surface and the second surface may comprise a convex surface relative to the optical axis of the lens. The concave surface may comprise an anterior curvature radius of the concave surface that is larger than a posterior curvature radius of the convex surface.

The power shape factor may be calculated according to the formula

$$\frac{{Power}_{ant} - {Power}_{post}}{{Power}_{ant} + {Power}_{post}} ,$$

wherein Powerₐₙₜ is the anterior surface power, and Powerₚₒₛₜ is the posterior surface power of the lens body.

The power shape factor may be calculated by ray tracing techniques applied to the intraocular lens. The ray tracing techniques may comprise utilizing a specific aperture size. The ray tracing techniques may comprise utilizing a specific aperture size, a best focus position and spherical aberration effects on the power shape factor.

A second embodiment of an intraocular lens comprises a lens body, wherein the lens body comprises: a refractive index between 1.50 and 1.60 inclusive, a diopter label power between 20 D and 28 D, inclusive, and a power shape factor that is less than or equal to −1.0 and greater than or equal to −3. Advantageously, providing an intraocular lens according to the label power range and power shape factor range of the second embodiment allows for correction of peripheral astigmatism in accordance with a blur parameter of less than 1.8 D, while optimizing on-axis visual acuity. Within these ranges, the power shape factor is stable, meaning that the structural features may be selected in order to produce lenses that are predictably capable of producing the effect of reducing off-axis astigmatism at 20 degree eccentricity while optimizing on-axis visual acuity. These ranges are critical for allowing predictable correction of peripheral astigmatism for patients having a dioptric power requirement that is less than a transition point power. The transition point power is the label power that corresponds to the shape factor of a plano-convex lens, which is −1.

The lens may comprise a central thickness that is between 0.45 mm and 1.0 mm. The intraocular lens may further comprise an anterior haptic connected to the lens body, and the lens may comprise a vault height that is between 0.30 mm and 0.65 mm. The lens may comprise a first surface having an anterior surface curvature greater than −0.022 mm⁻¹ and less than 0.00 mm⁻¹ and a second surface having a posterior surface curvature greater than −0.170 mm⁻¹ and less than −0.081 mm⁻¹. Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of the invention, to further aid in the ability of the lenses to predictably improve peripheral vision.

The lens may comprise a diopter label power that is between 20 D and 27 D. The power shape factor may be less than or equal to −1.0 and greater than or equal to −2.4. Advantageously, these ranges of label power and power shape factor are critical for achieving a lens that is optimized such that the lens minimizes off-axis astigmatism while maximizing on-axis visual acuity. Across these ranges, the power shape factor of the lens is particularly stable which allows for a greater ability to predict the structural features of the lens that result in optimization. This allows for optimization of the correction of peripheral vision for patients having a dioptric power requirement that is less than the transition point power. The lens may comprise a central thickness that is between 0.55 mm and 0.90 mm. An anterior haptic may be connected to the lens body, and wherein the lens may comprise a vault height that is between 0.35 mm and 0.60 mm. The lens may comprise a first surface having an anterior surface curvature greater than −0.022 mm⁻¹ and less than 0.00 mm⁻¹ and a second surface having a posterior surface curvature greater than −0.170 mm⁻¹ and less than −0.084 mm⁻¹. Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of optimization, to further aid in the ability of the lenses to predictably minimize peripheral vision while maximizing on-axis visual acuity.

The power shape factor may be calculated using paraxially defined curvature radii of the first surface and the second surface.

The first surface of the lens may comprise a concave surface and the second surface may comprise a convex surface relative to the optical axis of the lens. The concave surface may comprise an anterior curvature radius of the concave surface that is larger than a posterior curvature radius of the convex surface.

The power shape factor may be calculated according to the formula

$$\frac{{Power}_{ant} - {Power}_{post}}{{Power}_{ant} + {Power}_{post}} ,$$

wherein Powerₐₙₜ is the anterior surface power, and Powerₚₒₛₜ is the posterior surface power of the lens body.

The power shape factor may be calculated by ray tracing techniques applied to the intraocular lens. The ray tracing techniques may comprise utilizing a specific aperture size. The ray tracing techniques may comprise utilizing a specific aperture size, a best focus position and spherical aberration effects on the power shape factor.

A third embodiment of an intraocular lens comprises: a lens body, wherein the lens body comprises: a refractive index between 1.40 and 1.50 inclusive, a diopter label power between 23 D and 30 D, inclusive, and a power shape factor that is less than or equal to −0.2 and greater than or equal to −1. Advantageously, providing an intraocular lens according to the label power range and power shape factor range of the third embodiment allows for correction of peripheral astigmatism in accordance with a blur parameter of less than 1.8 D, while optimizing on-axis visual acuity. Within these ranges, the power shape factor is stable, meaning that the structural features may be selected in order to produce lenses that are predictably capable of producing the effect of reducing off-axis astigmatism at 20 degree eccentricity while optimizing on-axis visual acuity. The ranges of the third embodiment of the invention are critical for allowing for correction of peripheral astigmatism for patients having a dioptric power requirement greater than a transition point power. The transition point power is the label power that corresponds to the shape factor of a plano-convex lens, which is −1.

The lens may comprise a central thickness that is between 0.62 mm and 1.0 mm. An anterior haptic may be connected to the lens body, and the lens may comprise a vault height that is between 0.34 mm and 0.65 mm. The lens may comprise a first surface having an anterior surface curvature greater than 0 mm⁻¹ and less than 0.120 mm⁻¹ and a second surface having a posterior surface curvature greater than −0.367 mm⁻¹ and less than −0.130 mm⁻¹.

Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of the invention, to further aid in the ability of the lenses to predictably improve peripheral vision.

The lens may comprise a diopter label power that is between 24 D and 29 D, inclusive. The power shape factor is less than or equal to −0.3 and greater than or equal to −0.8. Advantageously, these ranges of label power and power shape factor are critical for resulting in a lens that is optimized such that the lens minimizes off-axis astigmatism while maximizing on-axis visual acuity. Across these ranges, the power shape factor of the lens is particularly stable which allows for a greater ability to predict the structural features of the lens that result in optimization for patients having a dioptric power requirement greater than the transition point power. The lens may comprise a central thickness that is between 0.70 mm and 0.90 mm. An anterior haptic may be connected to the lens body, and the lens may comprise a vault height that is between 0.40 mm and 0.60 mm. The lens may comprise a first surface having an anterior surface curvature greater than 0 mm⁻¹ and less than 0.12 mm⁻¹ and a second surface having a posterior surface curvature greater than −0.36 mm⁻¹ and less than −0.13 mm⁻¹. Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of minimization, to further aid in the ability of the lenses to predictably minimize peripheral vision while maximizing on-axis visual acuity.

The power shape factor may be calculated using paraxially defined curvature radii of the first surface and the second surface.

The power shape factor may be calculated according to the formula

$$\frac{{Power}_{ant} - {Power}_{post}}{{Power}_{ant} + {Power}_{post}} ,$$

wherein Powerₐₙₜ is the anterior surface power, and Powerₚₒₛₜ is the posterior surface power of the lens body.

The power shape factor may be calculated by ray tracing techniques applied to the intraocular lens. The ray tracing techniques may comprise utilizing a specific aperture size. The ray tracing techniques may comprise utilizing a specific aperture size, a best focus position and spherical aberration effects on the power shape factor.

A fourth embodiment of an intraocular lens comprises: a lens body, wherein the lens body comprises: a refractive index between 1.50 and 1.60 inclusive, a diopter label power between 23 D and 35 D, inclusive, and a power shape factor that is less than or equal to −0.2 and greater than or equal to −1. Advantageously, providing an intraocular lens according to the label power range and power shape factor range of the first embodiment allows for correction of peripheral astigmatism in accordance with a blur parameter of less than 1.8 D, while optimizing on-axis visual acuity. Within these ranges, the power shape factor is stable, meaning that the structural features may be selected in order to produce lenses that are predictably capable of producing the effect of reducing off-axis astigmatism at 20 degree eccentricity while optimizing on-axis visual acuity. The ranges of the fourth embodiment of the invention are critical for allowing for correction of peripheral astigmatism for patients having a dioptric power requirement greater than a transition point power. The transition point power is the label power that corresponds to the shape factor of a plano-convex lens, which is −1.

The lens may comprise a central thickness that is between 0.45 mm and 1.0 mm. An anterior haptic may be connected to the lens body, and the lens may comprise a vault height that is between 0.30 mm and 0.65 mm. The lens may comprise a first surface having an anterior surface curvature greater than 0.00 mm⁻¹ and less than 0.082 mm⁻¹ and a second surface having a posterior surface curvature greater than −0.179 mm⁻¹ and less than −0.081 mm⁻¹.

Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of the invention, to further aid in the ability of the lenses to predictably improve peripheral vision.

The intraocular lens may have a diopter label power between 25 D and 35 D, inclusive. The power shape factor may be less than or equal to −0.40 and greater than or equal to −0.95. Advantageously, these ranges of label power and power shape factor are critical for resulting in a lens that is optimized such that the lens minimizes off-axis astigmatism while maximizing on-axis visual acuity. Across these ranges, the power shape factor of the lens is particularly stable which allows for a greater ability to predict the structural features of the lens that result in optimization for patients having a dioptric power requirement greater than the transition point power. The lens may comprise a central thickness that is between 0.55 mm and 0.90 mm. An anterior haptic may be connected to the lens body, and the lens may comprise a vault height that is between 0.35 mm and 0.60 mm. The lens may comprise a first surface having an anterior surface curvature greater than 0.01 mm⁻¹ and less than 0.082 mm⁻¹ and a second surface having a posterior surface curvature greater than −0.179 mm⁻¹ and less than −0.081 mm⁻¹.

Advantageously, selecting any of these parameters to be in their respective ranges increases the adaptability of the lenses by allowing the lenses to be implanted with industrially available inserters. These ranges for each parameter also prevent the lenses from being excessively steep and thereby reduce discomfort in when inserted in a patient. The respective selection of these parameters further allows the lenses to have a power shape factor that is even more stable within the ranges of optimization, to further aid in the ability of the lenses to predictably minimize peripheral vision while maximizing on-axis visual acuity.

The power shape factor may be calculated using paraxially defined curvature radii of the first surface and the second surface.

The power shape factor may be calculated according to the formula

$$\frac{{Power}_{ant} - {Power}_{post}}{{Power}_{ant} + {Power}_{post}} ,$$

wherein Powerₐₙₜ is the anterior surface power, and Powerₚₒₛₜ is the posterior surface power of the lens body.

The power shape factor may be calculated by ray tracing techniques applied to the intraocular lens. The ray tracing techniques may comprise utilizing a specific aperture size. The ray tracing techniques may comprise utilizing a specific aperture size, a best focus position and spherical aberration effects on the power shape factor.

The invention also provides a set of intraocular lenses that improve peripheral vision; the set comprising: at least one lens according to the first embodiment and/or the second embodiment; and at least one lens according to the third embodiment and/or the fourth embodiment. The provision of a set of lenses allows for correction of peripheral vision for prescriptions requiring a dioptric power less than a transition point power, in addition to correction of peripheral vision for prescriptions having a dioptric power greater than a transition point power. The transition point power may be a label power corresponding to a shape factor of −1. The set of lenses of the invention therefore allows for correction of peripheral vision across the entire range of possible prescriptions and adaptation of the provided lens based on the prescription.

The at least one lens may comprise a series of lenses wherein the diopter label power of each lens of the series of lenses differs by at least 0.25 D. Advantageously, the set may provide a range of lenses that may be selected by a user to tailor the selected lenses to the patient's dioptric power requirements for those below the transition point power, and for those above the transition point power.

The invention also provides a method of designing a set of intraocular lenses for improved peripheral vision wherein as the label power of the lens increases from 17 D to 30 D, the power shape factor increases from −2.5 to −0.2, the method comprising:
for each label power increment of at least 0.25 D, providing an IOL having an optical power that reduces optical errors in an image produced at a peripheral retinal location of a patient's eye disposed at a distance from the fovea,
wherein for label powers having a shape factor less than or equal to −1, the IOL comprises a lens body formed according to the first embodiment and/or the second embodiment,
wherein for label powers having a shape factor greater than or equal to −1, the IOL comprises a lens body formed according to the third embodiment and/or the fourth embodiment,
wherein the blur parameter of each IOL is less than 1.8 diopters, wherein the blur parameter is calculated according to: Blur Parameter=\sqrt{Sphere²+Cylinder²}. The provision of a set of lenses designed according to the method of the invention allows for correction of peripheral vision for prescriptions requiring a dioptric power less than a transition point power, in addition to correction of peripheral vision for prescriptions having a dioptric power greater than a transition point power. The transition point power is a label power corresponding to a shape factor of −1. The set of lenses of the invention therefore allows for correction of peripheral vision across the entire range of possible prescriptions and adaptation of the provided lens based on the prescription.

### BRIEF DESCRIPTION OF THE DRAWINGS

FIG. 1 illustrates a prior art example of using an intraocular lens with a concave anterior surface and convex posterior surface to adjust the focal length of peripheral images focusing on a retina of an eye.

FIG. 2 is an illustration of an intraocular lens according to some embodiments of this disclosure.

FIG. 3 is a graphical representation and corresponding formulas for calculating surface power values from respective refractive indices and curvature radii according to embodiments of this disclosure.

FIG. 4 is a graphical representation of peripheral vision lenses having a refractive index of 1.471.

FIG. 5 is a graphical representation of peripheral vision lenses having a refractive index of 1.55.

FIG. 6 is a graphical representation of a region of shape factors and label powers in which lenses having a refractive index of 1.471 have been optimized according to the discussion herein.

FIG. 7 is a graphical representation of a region of shape factors and label powers in which lenses having a refractive index of 1.55 have been optimized according to the discussion herein.

### DETAILED DESCRIPTION

This disclosure considers multiple kinds of ophthalmic lenses used to correct vision and presents reliable solutions to problems involved when an intraocular lens is to be implanted in a patient and provides reliable positioning for structural features of IOLs of different label powers.

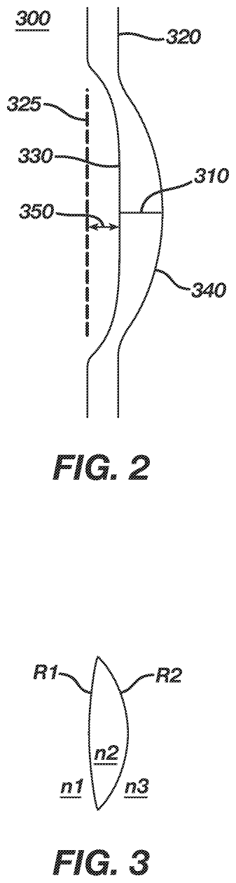

Embodiments of this disclosure incorporate the plain meaning of terms of art in the field of IOLs. For example, as shown in FIG. 2, this disclosure refers to the haptics 320 attached to a lens body that are equipped to hold the IOL 300 in place in a patient's eye. The haptics 320 establish an anterior haptics plane 325 that is a useful reference point for comparative purposes of different lens. An anterior haptics plane 325 is defined by a plane that is normal to the optical axis and extending across the front-most surface of the uncompressed IOL haptics 320. As shown in FIG. 2, a vault height 350 of an IOL is the distance between the anterior haptics plane 325, and a plane normal to the optical axis containing an anterior surface 330 of the IOL body, calculated as defined in ISO standard 11979-1. FIG. 2 also shows a measurement of a central thickness 310, which is the difference between the sagittal thickness and the vault height as defined by ISO standard 11979-1. The intraocular lens body of FIG. 2 has an anterior surface 330, a posterior surface 340, a front principal plane determined, at least in part, by the optical effects of the anterior surface of the IOL 300 and a back principal plane determined, at least in part, by the optical effects of the posterior surface of the IOL 300. This disclosure calculates the best possible structure for a given label power measured in diopters. As used herein, “label power” refers to the dioptric power as defined in ISO standard 11979-1 which may be calculated using ray tracing techniques. Central thickness 310, anterior surface curvature, and posterior surface curvature are used in accordance with standards in the art of intraocular lenses. Standard materials for forming IOLs are within the scope of this disclosure. In various implementations, the optic can comprise materials such as acrylic, silicone, polymethylmethacrylate (PMMA), block copolymers of styrene-ethylene-butylene-styrene (C-FLEX) or other styrene-base copolymers, polyvinyl alcohol (PV A), polystyrenes, polyurethanes, hydrogels, etc.

This disclosure uses power shape factor values for intraocular lenses (IOLs) to identify structural features of IOLs necessary to achieve different label powers measured in diopters (D) that are appropriate upon implantation in patients. By managing and engineering at least the structural features disclosed herein and shown in FIG. 2 and equations 1 to 10 below, the IOLs of this disclosure perform extremely well in treating eye disease and maintaining focus and clarity for both foveal vision and peripheral vision for a patient receiving an implanted IOL.

As an initial introduction, this disclosure uses at least two different IOL structures that are configured as peripheral vision correcting IOLs as non-limiting examples showing the features and benefits of the work disclosed herein. FIG. 2 illustrates a lens in which the concave surface faces the object to be imaged and/or the light source of the image. An IOL may have convex surfaces for both the anterior and posterior sides of the IOL. Plano-convex IOLs have an anterior surface that is flat or substantially flat and a convex posterior surface.

The power shape factor varies for different kinds of lenses but may also be constant for a lens having one planar surface. For example, a constant power shape factor is equal to negative one (−1) for a plano-convex lens. In non-limiting embodiments, IOLs with power shape factors that are less than or equal to minus one (−1) are provided, and IOLs with power shape factors that are greater than or equal to minus one (−1) are also provided. An IOL having a power shape factor greater than or equal to minus one may have a lens body that is formed differently from the lens body of an IOL having a power shape factor less than or equal to minus one. Without limiting this disclosure to any particular power shape factors or label powers, the lenses of this disclosure may have a range of label powers from about 17 D to 35 D to illustrate various embodiments. At a constant power shape factor of a plano-convex lens (e.g., minus 1), the lens shape transitions from that of a lens having a power shape factor less than or equal to minus one to that of a lens having a power shape factor greater than or equal to minus one. At lower label power ranges, including but not limited to a range from 7 D to 17 D, the IOL anterior and posterior surfaces do not necessarily exhibit power responses that would be expected from simple surfaces with consistent curvature across the anterior surface and the posterior surface. Instead the anterior surface and the posterior surface of the IOL may effectively include complex surfaces that vary across the respective surface. The variance across the surfaces may be in terms of surface curvature variations (i.e., exhibiting toroidal surface structures or Zernike surfaces) and may require further investigation to ensure that a desired, reliable response is provided upon IOL implantation in a patient.

The power shape factor of an IOL is calculated according to the formula:

$$\frac{{Power}_{ant} - {Power}_{post}}{{Power}_{ant} + {Power}_{post}} , \qquad ( Eq . 1 )$$

wherein Powerₐₙₜ is the anterior surface power, and Powerₚₒₛₜ is the posterior surface power of the lens body.

Anterior surface power is calculated according to:

$${Power}_{ant} = \frac{{n}_{IOL} - {n}_{media}}{{R}_{ant}} \qquad ( Eq . 2 )$$

Posterior surface power is calculated according to:

$${Power}_{post} = \frac{{n}_{media} - {n}_{IOL}}{{R}_{post}} \qquad ( Eq . 3 )$$

wherein Rₐₙₜ is an anterior curvature radius, and Rₚₒₛₜ is a posterior curvature radius of the lens body. A first surface of an IOL defines the anterior curvature radius Rₐₙₜ and a second surface defines the posterior curvature radius Rₚₒₛₜ: n_{IOL} is the refractive index of the IOL, while n_{media} is the refractive index of the media in which the IOL is implanted. For power in Diopters, the radii must be given in millimeters.

The radius shape factor for an IOL is calculated according to the formula

$$( \frac{{R}_{post} - {R}_{ant}}{{R}_{post} + {R}_{ant}} ) . \qquad ( Eq . 4 )$$

where the radius shape factor utilizes the overall label radius of curvature for each lens, the power shape factor may be calculated by ray tracing techniques applied to the intraocular lens. In some non-limiting embodiments, the ray tracing techniques may include utilizing a specific aperture size. The ray tracing techniques may also combine utilizing a specific aperture size, a best focus position and spherical aberration effects on the power shape factor. The power shape factor and the radius shape factor may be equal for ideally simple surfaces of known curvature, but the values are not always the same, indicating more complicated surface curvatures.

The lens maker formula illustrates how an intraocular lens focal length is affected by the radius of curvature of both the anterior surface and the posterior surface, where F is the focal length, now is the refractive index of the lens, R₁ is the radius of curvature of the first surface and R₂ is the radius of curvature of the second surface:

$$\frac{1}{F} = ( {n}_{IOL} - 1 ) ( \frac{1}{{R}_{1}} - \frac{1}{{R}_{2}} ) \qquad ( Eq . 5 )$$

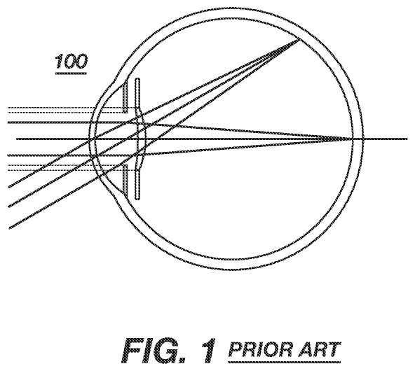

Prior art efforts, such as the IOL 100 shown in FIG. 1, have focused on the lens makers equation to adjust radius values and focal lengths for the IOLs with no deep analysis of the variable power responses that anterior surfaces and posterior surfaces may exhibit upon implantation.

Measurements given throughout this application are discussed in relation to the Liou and Brennan eye model (Liou H L, Brennan N A. Anatomically accurate, finite model eye for optical modeling. J Opt Soc Am A Opt Image Sci Vis. 1997 August; 14 (8): 1684-95. doi: 10.1364/josaa.14.001684. PMID: 9248060). For lenses of this disclosure, the ranges of vault height are chosen between 0.34 mm and 0.65 mm. Lenses may comprise vault height in the range of 0.40 mm to 0.60 mm, or 0.50 mm to 0.55 mm. The vault height may be 0.65 mm. The central thickness is chosen in the range from 0.62 mm to 1.0007 mm. The central thickness may range from 0.70 mm to 0.90 mm, or from 0.80 mm to 0.85 mm. The shape factor may be in the range −4 to 0. These ranges of parameters are selected to provide lenses that may be implanted with industry available inserters, as well as to avoid producing lenses that are excessively steep to thereby reduce discomfort in a patient. For these reasons, lenses having a posterior radius lower than −4 mm are also excluded from this disclosure. In this disclosure, eye length is fixed by the paraxial focus of an equivalent ZCB00 (Tecnis® monofocal) lens having a power 2D below the label power of the IOL (i.e., an IOL having a label power of 22 D uses the eye length corresponding to a 20 D ZCB00 lens).

An implanted IOL may be classified as a lens that corrects peripheral vision if, for the implanted IOL, the off-axis astigmatism measured at 20 degree eccentricity as measured from the optical axis through the iris is less than or equal to a threshold value of 1.8 D. Eccentricity refers to the angular distance from the center of the visual field, such as the central fovea. The off-axis astigmatism at 20 degrees may be calculated according to a peripheral blur parameter of an IOL as measured at that angle. For example, for a IOL having a label power of 20 D that corrects peripheral vision, the peripheral astigmatism may be 1.3 D or 1.7 D at 20 degree eccentricity.

The vectors J₀ and J₄₅ as well as sphere, cylinder and blur parameter may be calculated. These values are calculated according to the following equations, in which C(i, j) are corresponding Zernike coefficients in μm and r is the radius of the pupil in mm. For these formulae, a negative sign convention is used for the definition of cylinder and the retinal curvature is defined according to Atchinson et al. (Optical models for human myopic eyes, 2006).

$$\begin{matrix}{J}_{0} = - \frac{2 \sqrt{6} \cdot C ( 2 , 2 )}{{r}^{2}} & ( Eq . 6 ) \\ {J}_{45} = - \frac{2 \sqrt{6} \cdot C ( 2 , - 2 )}{{r}^{2}} & ( Eq . 7 ) \\ Sphere = - \frac{4 \sqrt{3} \cdot C ( 2 , 0 )}{{r}^{2}} & ( Eq . 8 ) \\ Cylinder = - 2 \cdot \sqrt{{J}_{0}^{2} + {J}_{45}^{2}} & ( Eq . 9 ) \\ Blur Parameter = \sqrt{{Sphere}^{2} + {Cylinder}^{2}} & ( Eq . 10 )\end{matrix}$$

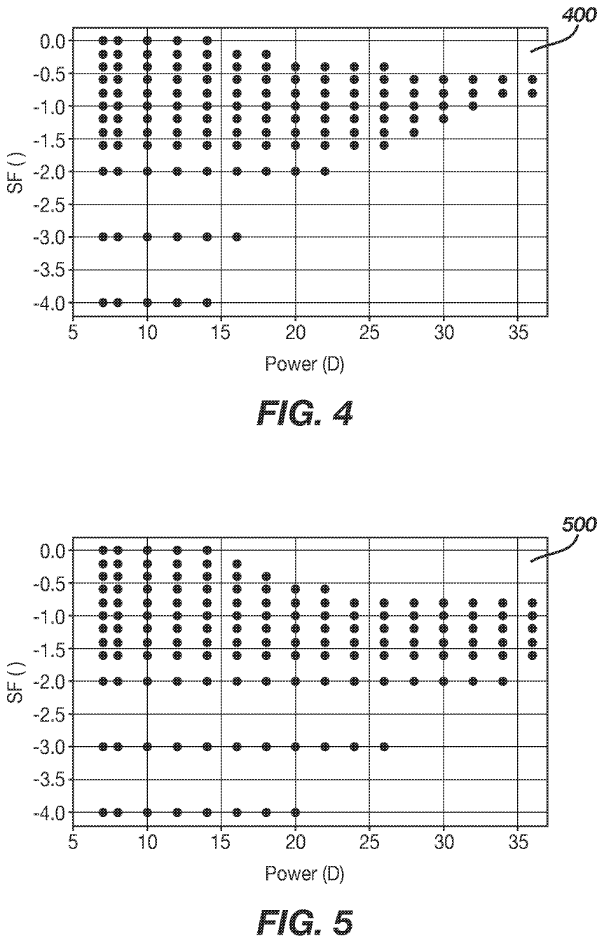

With reference to FIGS. 4 and 5, this disclosure provides ranges that are critical for lenses having label powers that correct peripheral vision according to the blur parameter condition, have an absolute value below 1.8 D for peripheral cylinder at 20 degree eccentricity, and optimize on-axis visual acuity and contrast sensitivity. On-axis visual acuity and contrast sensitivity are optimized for selected values of vault height, central thickness and shape factor within the ranges discussed herein. For the selected values, the on-axis visual acuity and contrast sensitivity are optimized by adjusting the anterior and/or posterior aspheric parameters to produce a lens having an on-axis performance that is as close as possible to a diffraction limited performance for an entrance pupil of 5.65 mm on axis in green light (550 nm). Graph 400 of FIG. 4 shows peripheral vision lenses having a refractive index of 1.471 that correct peripheral vision according to the blur parameter condition, have an absolute value below 1.8 D for peripheral cylinder at 20 degree eccentricity, and optimize on-axis visual acuity and contrast sensitivity, while graph 500 of FIG. 5 shows peripheral vision lenses having a refractive index of 1.55 that correct peripheral vision according to the blur parameter condition, have an absolute value below 1.8 D for peripheral cylinder at 20 degree eccentricity, and optimize on-axis visual acuity. Both FIG. 4 and FIG. 5 respectively show plots of shape factor against label power for lenses meeting the conditions. In both plots, the lenses are constrained by having a vault height between 0.34 mm and 0.65 mm, and central thickness the range from 0.62 mm to 1.0007 mm.

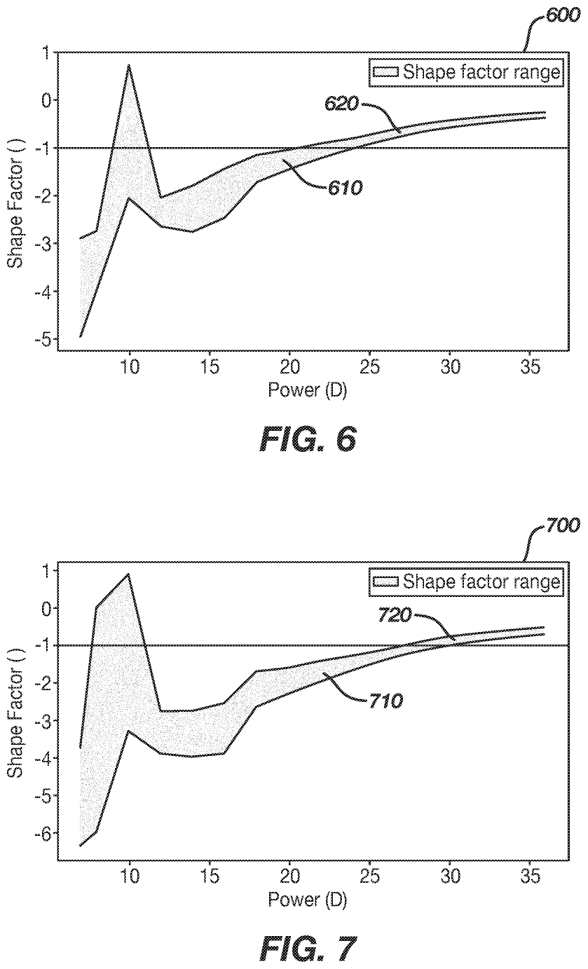

With reference to graphs 600 and 700 of FIGS. 6 and 7, respectively, this disclosure also provides sub-ranges corresponding to optimal label powers and power shape factors that are critical for a peripheral vision lens that maximizes the reduction of off-axis astigmatism at 20 degree eccentricity, while maximizing the on-axis visual acuity and contrast sensitivity. FIG. 6 shows the optimal region for a refractive index of 1.471 as the shaded regions 610 and 620. FIG. 7 shows the optimal region for a refractive index of 1.55 as the shaded regions 710 and 720. In both graphs, the lenses are constrained by having a vault height between 0.34 mm and 0.65 mm, and central thickness the range from 0.62 mm to 1.0007 mm.

In order to calculate these sub-ranges corresponding to regions 610, 620, 710 and 720, an optimization of a modulation transfer function (MTF) was carried out. It is known that contrast sensitivity is proportional to Modulation Transfer Function (MTF); thus, sufficient on-axis visual acuity and/or contrast sensitivity can be achieved through optimizing MTF. Optimization can be done at one or several spatial frequencies, or using a figure of merit proportional to MTF levels at one or several spatial frequencies. In a non-limiting example, sufficient contrast sensitivity is achieved if MTF is at least 0.7 for a 5 mm pupil in green light at a spatial frequency of 50 cycles/mm, as measured in Eye model #2 according to ISO 11979-2 2014. The following parameters and their associated weighting for the MTF optimization are as follows: a lens that is on-axis diffraction limited for a large 5.65 mm entrance pupil (weight=1); reducing each of off-axis astigmatism and off-axis defocus at 20 degrees for an entrance pupil of 4 mm (weight=0.01 each); increasing off-axis 20 degree MTF values for each of sagittal and tangential foci at a spatial frequency of 25 cycles per mm for an entrance pupil of 4 mm (weight=0.02 each); off-axis 20 degree MTF values for sagittal and tangential foci at a spatial frequency of 25 cycles per mm for an entrance pupil of 4 mm that are as similar as possible (weight=0.01). The MTF was calculated using monochromatic green light at 550 nm, with the lens anterior haptics plane about 0.5 mm behind the iris in a Liou and Brennan eye model, and with retinal curvature defined according to Atchinson et al. (Optical models for human myopic eyes, 2006). This optimization may be used to determine how parameters of a lens, such as vault height, central thickness, anterior surface curvature and posterior surface curvature and shape factor, may be tailored (in accordance with the lens maker's equation) to achieve desirable sub-ranges of label powers that are optimal for improving the peripheral vision of a patient. The sub-ranges were then selected according to the region over which the observed optimization is most stable, so that the optimization effect may be predictable given appropriate selections of structural features.

FIG. 3 sets forth an example of features of IOLs by taking into account the respective refractive indices for the media before the lens (n1), the lens material (n2), and the media after the lens (n3). With known curvatures of radius (R₁, R₂) for the anterior lens surface and the posterior lens surface, the surface powers (i.e., Powerₐₙₜ and Powerₚₒₛₜ) of the lens can be calculated according to equations 2 and 3 given above. For Powerₐₙₜ, n_{media} corresponds to n₁, while for Powerₚₒₛₜ, n_{media} corresponds to n₂. This disclosure includes surface curvature values across numerous diopter label powers for the anterior surfaces and posterior surfaces of numerous different lenses having respective refractive indices. This disclosure considers an IOL having a power shape factor less than or equal to minus one and/or an IOL having a power shape factor greater than or equal to minus one. The lens body of the IOL having a power shape factor less than or equal to minus one may be formed differently from the lens body of the IOL having a power shape factor greater than or equal to minus one. The disclosure may provide a set of lenses comprising at least one first lens having a power shape factor less than or equal to minus one. Additionally, or alternatively, the set of lenses may comprise at least one second lens having a power shape factor greater than or equal to minus one. The lens having a power shape factor less than or equal to minus one and/or the lens having a power shape factor greater than or equal to minus one may have a refractive index (n2) between 1.40 and 1.50 and/or a refractive index (n2) between 1.50 and 1.60. Other refractive index ranges are considered. The refractive index may be between 1.45 and 1.48. The refractive index may be between 1.54 and 1.56. The refractive index may be 1.471. The refractive index may be 1.55. The respective power shape factors, and radius of curvature for different refractive indices coincide with the patterns established for the above discussed lenses. In fact, the differences and similarities between power shape factors for the above discussed lenses indicate patterns that are useful to predict effects of lens features such as but not limited to vault height as discussed above. These same effects may be shown in further testing for lenses having variable refractive index values as disclosed herein.

This disclosure sets forth structural factors for IOLs that have shown exceptional results for foveal vision and peripheral vision upon implantation in accordance with the optimization discussed herein.

Lenses Having a Power Shape Factor Less than or Equal to Minus One

As noted above, and as demonstrated in FIGS. 4 to 7, certain lower label powers, for example label powers for a lens having a power shape factor less than or equal to minus one, exhibit power shape factor values that are less stable across the range of lower label powers. The range of lower label powers may be from 7 D to 17 D. As further shown by FIGS. 4 to 7, similar uncertainty is seen at higher label powers above 28 D and below 36 D for a lens having a power shape factor less than or equal to minus one. This provides a basis for fine tuning the anterior surface curvature and posterior surface curvature of each lens at each label power. Due to the selection of central thickness, vault height and shape factor in the ranges described in this disclosure, anterior surface curvature and posterior surface curvature can be adjusted to produce peripheral vision lenses having a stable power response over a range of label powers. Demonstrated by graph 400 of FIG. 4, the range in which the power response is stable for an IOL having a power shape factor less than or equal to minus one and a refractive index between 1.40 and 1.50 is 17 D to 23 D. With reference to graph 500 of FIG. 5, for an IOL having a power shape factor less than or equal to minus one and a refractive index between 1.50 and 1.60, the stable range of label powers is between 20 D and 28 D. Lenses having these label ranges have a readily predictable ability to optimize on-axis visual acuity to be as close as possible to the boundary of diffraction limited performance, while correcting peripheral vision.

An embodiment of a peripheral vision IOL comprises a lens body. The lens body has a diopter label power between 17 D and 23 D and a refractive index value between about 1.40 and 1.50. For these label powers and refractive indices, the power shape factor is between −1 and −2.5. The first surface may have the anterior surface curvature between −0.77 mm⁻¹ and 0.00 mm⁻¹. The second surface may have the posterior surface curvature between −0.355 mm⁻¹ and −0.130 mm⁻¹. The central thickness may be between 0.62 mm and 1.0 mm. An anterior haptic may be connected to the lens body to hold the lens in place after implantation and the IOL may exhibit a vault height between 0.34 mm and 0.65 mm.

For an embodiment of a lens body having a refractive index between about 1.50 and 1.60 and diopter label powers between 20 D and 28 D, the power shape factor is between −1 and −3. The first surface may have the anterior surface curvature between −0.022 mm⁻¹ and 0.00 mm⁻¹. The second surface may have the posterior surface curvature between −0.170 mm⁻¹ and −0.081 mm⁻¹. The central thickness may be between 0.45 mm and 1.0 mm. An anterior haptic may be connected to the lens body to hold the lens in place after implantation and the IOL may exhibit a vault height between 0.30 mm and 0.65 mm.

An IOL comprising a lens body made according to the following sub-ranges of label power and power shape factor may be provided. The sub-range of label power may be 18 D and 22 D for a lens having a refractive index between about 1.40 and 1.50, and between 20 D and 27 D for a lens having a refractive index between 1.50 and 1.60. With reference to the optimization function of FIGS. 6 and 7, the narrow bands 610 and 710 of the optimization function within these sub-ranges demonstrates that lenses made having label powers within the sub-ranges have an even more stable shape factor across the range, in comparison to the wider ranges discussed above. This stability means that it is easier to predict which structural features will result in lenses that are capable of maximizing on-axis visual acuity while minimizing off-axis astigmatism at 20 degree eccentricity in accordance with the optimization MTF described in this application.

In an embodiment, the lens has a diopter label power between 18 D and 22 D and refractive index values between about 1.40 and 1.50. The lens has a power shape factor between −1.1 and −2.0. In this case, the first surface may have an anterior surface curvature, or between −0.071 mm⁻¹ and −0.00 mm⁻¹. The second surface may have a posterior surface curvature between −0.355 mm⁻¹ and −0.134 mm⁻¹. The central thickness for the lens may be between 0.70 mm and 0.90 mm. An anterior haptic may be connected to the lens body to hold the lens in place after implantation and the IOL may exhibit a vault height between 0.40 mm and 0.60 mm.

A further embodiment of a lens has a diopter label power between 20 D and 27 D and a refractive index between about 1.50 and 1.60. In this case, the lens has a power shape factor between −1.0 and −2.4. The first surface may have an anterior surface curvature between −0.022 mm⁻¹ and −0.00 mm⁻¹. The second surface may have a posterior surface curvature between −0.170 mm⁻¹ and −0.084 mm⁻¹. The central thickness may be between 0.55 mm and 0.90 mm. An anterior haptic may be connected to the lens body to hold the lens in place after implantation and the IOL may exhibit a vault height between 0.35 mm and 0.60 mm.

Lenses Having a Power Shape Factor Greater than or Equal to Minus One

As discussed above, for an IOL having a label power greater than a transition point power, the intraocular lens includes a lens body that may be formed differently from the lens body for an IOL having a label power less than a transition point power. The transition point power may be a label power that corresponds to an IOL having a shape factor of −1. For example, with reference to FIG. 4, the transition point power may be 23 D, 24 D or 25 D for a lens having a refractive index between 1.40 and 1.50. With reference to FIG. 5, for a lens having a refractive index between 1.50 and 1.60, the transition point power may be 23 D, 25 D or 28 D. Other transition point powers may be chosen as appropriate.

For higher values of label powers for lenses having a power shape factor greater than or equal to minus one, the rate of increase of power shape factors across the ranges of label powers diverges but to a smaller extent than previously discussed for label powers lower than the transition point power. With reference to FIG. 4, between certain increasing label powers, the rate of increase of the power shape factors across the range of label powers exhibits little variation. These ranges of label power may be between 23 D and 30 D for a lens having a power shape factor greater than or equal to minus one and a refractive index between 1.40 and 1.50 or between 23 D and 35 D for a lens having a power shape factor greater than or equal to minus one and a refractive index between 1.50 and 1.60. Within these ranges, the anterior surface curvature and the posterior surface curvature may be refined to produce readily predictable variation in shape factor across the whole range. This stability increases the ability to predict the structural features that allow the lenses to correct peripheral vision while optimizing on-axis visual acuity.

An embodiment of the lens has a diopter label power between 23 D and 30 D and refractive index values between about 1.40 and 1.50. The power shape factor is between −0.2 and −1. For these ranges, the first surface may have the anterior surface curvature between 0 mm⁻¹ and 0.120 mm⁻¹. The second surface may have the posterior surface curvature between −0.367 mm⁻¹ and −0.130 mm⁻¹. The central thickness may be between 0.62 mm and 1.0 mm. An anterior haptic may be connected to the lens body to hold the lens in place after implantation and the IOL may exhibit a vault height between 0.34 mm and 0.65 mm.

An embodiment of a lens having a refractive index between about 1.50 and 1.60 has diopter label powers between 23 D and 35 D. The power shape factor is between −0.2 and −1. In this case, the first surface may have the anterior surface curvature between 0.00 mm⁻¹ and 0.082 mm⁻¹. The second surface may have the posterior surface curvature between −0.179 mm⁻¹ and −0.081 mm⁻¹. The central thickness may be between 0.45 mm and 1.0 mm. An anterior haptic may be connected to the lens body to hold the lens in place after implantation and the IOL may exhibit a vault height between 0.30 mm and 0.65 mm.

Within at least one example sub-range, such as between 24 D and 29 D for a lens having a power shape factor greater than or equal to minus one having a refractive index between 1.40 and 1.50 as shown in area 620 of FIG. 6, or between 25 D and 35 D for a lens having a refractive index between 1.50 and 1.60 as shown in area 720 of FIG. 7, the rate of increase of the power shape factors is determined such that the differences are statistically insignificant and the power shape factor appears to exhibit even greater stability. These sub-ranges may be selected in order to optimize the structural features of the IOL to result in minimized off-axis astigmatism at 20 degree eccentricity and a maximized on-axis visual acuity to thereby improve peripheral vision. An IOL comprising a lens body, made according to the following sub-ranges of label power and power shape factor may be provided in order to maximize on-axis visual acuity while minimizing off-axis astigmatism at 20 degree eccentricity in accordance with this optimization disclosed herein.

A further embodiment of a lens has a diopter label power between 24 D and 29 D, a refractive index between about 1.40 and 1.50, and a power shape factor between −0.3 and −0.8. In this case, the first surface may have an anterior surface curvature between 0 mm⁻¹ and 0.12 mm=1. The second surface may have a posterior surface curvature between 0.36 mm⁻¹ and 0.13 mm⁻¹. The central thickness for the lens may be between 0.70 mm and 0.90 mm. An anterior haptic may be connected to the lens body to hold the lens in place after implantation and the IOL may exhibit a vault height between 0.40 mm and 0.60 mm.

An embodiment of the lens having a diopter label power between 25 D and 35 D and refractive index values between about 1.50 and 1.60 has a power shape factor between −0.7 and −0.95. In this case, the first surface may have an anterior surface curvature between 0.01 mm⁻¹ and 0.082 mm⁻¹. The second surface may have a posterior surface curvature between −0.179 mm⁻¹ and −0.081 mm⁻¹. The central thickness for the lens may be between 0.55 mm and 0.90 mm. An anterior haptic may be connected to the lens body to hold the lens in place after implantation and the IOL may exhibit a vault height between 0.35 mm and 0.60 mm.

### Set of Lenses

A set of lenses may be provided, the set of lenses comprising at least one lens having a power shape factor less than or equal to minus one as defined above, and/or at least one lens having a power shape factor greater than or equal to minus one as defined above. The set of lenses may comprise a series of lenses. The diopter label power of each lens in the series of lenses may vary by at least 0.25 D in comparison to the other lenses in the series of lenses. For example, the series of lenses may comprise 5 lenses having diopter label powers of 17.5 D, 17.75 D, 18 D, 18.25 D, 18.5 D. The diopter label power of each lens in the series of lenses may vary by 0.5 D, 1 D, 2 D, 3 D, 4 D or 5 D in comparison to the other lenses in the series of lenses. The lenses in the set of lenses may have a label power below a transition point power, or a label power above a transition point power. The lens body of the IOL having a label power above the transition point power may be formed differently from the lens body of the IOL having a label power below the transition point power. The transition point power is a label power that corresponds to an IOL having a shape factor of −1. For example, with reference to FIG. 4, the transition point power may be 23 D, 24 D or 25 D for a lens having a refractive index between 1.40 and 1.50. With reference to FIG. 5, for a lens having a refractive index between 1.50 and 1.60, the transition point power may be 23 D, 25 D or 28 D. Other transition point powers may be chosen as appropriate.

The provision of a set of lenses allows for correction of peripheral vision for prescriptions requiring a dioptric power less than a transition point power, in addition to correction of peripheral vision for prescriptions having a dioptric power greater than a transition point power. The embodiments of the set of lenses of the invention therefore allow for correction of peripheral vision across the entire range of possible prescriptions. Advantageously, the set may provide a range of lenses that may be selected by a user to tailor the selected lenses to the patient's dioptric power requirements.

In accordance with areas 610 and 620 on FIG. 6, an embodiment of a set of lenses having a refractive index between 1.40 and 1.50 comprises at least one first lens having a diopter label power of between 17 Diopters and 23 Diopters with a power shape factor that is less than or equal to −1.0 and greater than or equal to −2.5, and at least one second lens having a diopter label power of between 23 Diopters and 30 Diopters and with a power shape factor that is less than or equal to −0.2 and greater than or equal to −1. Within these ranges, the anterior surface curvature and the posterior surface curvature may be refined to produce readily predictable variation in shape factor across the whole range. This stability increases the ease with which lenses are capable of correcting peripheral vision while optimizing on-axis visual acuity.

For the at least one first lens, the first surface may have the anterior surface curvature between −0.77 mm⁻¹ and 0.00 mm⁻¹. The second surface may have the posterior surface curvature between −0.355 mm⁻¹ and −0.130 mm⁻¹. The central thickness may be between 0.62 mm and 1.0 mm. An anterior haptic may be connected to the lens body to hold the lens in place after implantation and the IOL may exhibit a vault height between 0.34 mm and 0.65 mm.

For the at least one second lens in the set of lenses, the first surface may have the anterior surface curvature between 0 mm⁻¹ and 0.120 mm⁻¹. The second surface may have the posterior surface curvature between −0.367 mm⁻¹ and −0.130 mm⁻¹. The central thickness may be between 0.62 mm and 1.0 mm. An anterior haptic may be connected to the lens body to hold the lens in place after implantation and the IOL may exhibit a vault height between 0.34 mm and 0.65 mm.

In accordance with areas 710 and 720 on FIG. 7, a second embodiment of a set of lenses comprises lenses having a refractive index between 1.50 and 1.60. The set of lenses comprises at least one first lens having a diopter label power of between 20 D and 28 D with a power shape factor between −1 and −3, and at least one second lens having a diopter label power of between 23 D and 35 D and with a power shape factor that is between −0.5 and −1. Within these ranges, the anterior surface curvature and the posterior surface curvature may be refined to produce readily predictable variation in shape factor across the whole range. This stability allows for greater predictability when producing lenses that are capable of correcting peripheral vision while optimizing on-axis visual acuity.

For the at least one first lens in the second embodiment of the set of lenses, the first surface may have the anterior surface curvature between −0.022 mm⁻¹ and 0.00 mm⁻¹. The second surface may have the posterior surface curvature between −0.170 mm⁻¹ and −0.081 mm⁻¹. The central thickness may be between 0.45 mm and 1.0 mm. An anterior haptic may be connected to the lens body to hold the lens in place after implantation and the IOL may exhibit a vault height between 0.30 mm and 0.65 mm.

For the at least one second lens in the second embodiment of the set of lenses, the first surface may have the anterior surface curvature between 0.00 mm⁻¹ and 0.082 mm⁻¹. The second surface may have the posterior surface curvature between −0.179 mm⁻¹ and −0.081 mm⁻¹. The central thickness may be between 0.45 mm and 1.0 mm. An anterior haptic may be connected to the lens body to hold the lens in place after implantation and the IOL may exhibit a vault height between 0.30 mm and 0.65 mm.

A third embodiment of a set of lenses comprises lenses having a refractive index between 1.40 and 1.50. The set of lenses comprises at least one first lens having a diopter label power of between 18 D and 22 D with a power shape factor between −1.1 and −2.0, and at least one second lens having a diopter label power between 24 D and 29 D and with a power shape factor that is between −0.3 and −0.8. The lenses within this set of lenses comprise sub-ranges of label power and shape factor that may be selected in order to optimize the structural features of the IOL to result in minimized off-axis astigmatism at 20 degrees and a maximized on-axis visual acuity to thereby improve peripheral vision.

The at least one first lens of the third embodiment of the set of lenses may have a first surface having an anterior surface curvature, or between −0.071 mm⁻¹ and −0.00 mm⁻¹. The second surface may have a posterior surface curvature between −0.355 mm⁻¹ and −0.134 mm⁻¹. The central thickness for the lens may be between 0.7 mm and 0.9 mm. An anterior haptic may be connected to the lens body to hold the lens in place after implantation and the IOL may exhibit a vault height between 0.40 mm and 0.60 mm.

The at least one second lens of the third embodiment of the set of lenses may have a first surface may having an anterior surface curvature between 0 mm⁻¹ and 0.12 mm=1. The second surface may have a posterior surface curvature between 0.36 mm⁻¹ and 0.13 mm⁻¹. The central thickness for the second lens may be between 0.7 mm and 0.9 mm. An anterior haptic may be connected to the lens body to hold the lens in place after implantation and the IOL may exhibit a vault height between 0.40 mm and 0.60 mm.

The fourth embodiment of a set of lenses comprises lenses having a refractive index between 1.50 and 1.60. The set of lenses comprises at least one first lens having a diopter label power of between 20 D and 27 D with a power shape factor between −1.0 and −2.4, and at least one second lens having a diopter label power between 25 D and 35 D and with a power shape factor that is between −0.7 and −0.95. The lenses within this set of lenses comprise sub-ranges of label power and shape factor that may be selected in order to optimize the structural features of the IOL to result in minimized off-axis astigmatism at 20 degrees and a maximized on-axis visual acuity to thereby improve peripheral vision.

For the first lens of the fourth embodiment, the first surface may have an anterior surface curvature between −0.022 mm⁻¹ and −0.00 mm⁻¹. The second surface may have a posterior surface curvature between −0.170 mm⁻¹ and −0.084 mm⁻¹. The central thickness for the first lens may be between 0.55 mm and 0.90 mm. An anterior haptic may be connected to the lens body to hold the lens in place after implantation and the IOL may exhibit a vault height between 0.35 mm and 0.60 mm.

For the at least one second lens of the fourth embodiment, the first surface may have an anterior surface curvature between 0.01 mm⁻¹ and 0.082 mm⁻¹. The second surface may have a posterior surface curvature between −0.179 mm⁻¹ and −0.081 mm⁻¹. The central thickness for the lens may be between 0.55 mm and 0.90 mm. An anterior haptic may be connected to the lens body to hold the lens in place after implantation and the IOL may exhibit a vault height between 0.35 mm and 0.60 mm.

A fifth embodiment of a set of lenses comprises at least one first lens having a refractive index between 1.40 and 1.50, a diopter label power of between 17 D and 23 D with a power shape factor that is less than or equal to −1.0 and greater than or equal to −2.5, and at least one second lens having a refractive index between 1.50 and 1.60, a diopter label power of between 23 D and 35 D and with a power shape factor that is between −0.5 and −1. Within these ranges, the anterior surface curvature and the posterior surface curvature may be refined to produce readily predictable variation in shape factor across the whole range. This stability allows for greater predictability when producing lenses that are capable of correcting peripheral vision while optimizing on-axis visual acuity.

A sixth embodiment of a set of lenses comprises at least one first lens having a refractive index between 1.50 and 1.60, a diopter label power of between 20 D and 28 D and a power shape factor between −1 and −3, and at least one second lens having a refractive index between 1.40 and 1.50 and a diopter label power of between 23 Diopters and 30 Diopters and with a power shape factor that is less than or equal to −0.2 and greater than or equal to −1. Within these ranges, the anterior surface curvature and the posterior surface curvature may be refined to produce readily predictable variation in shape factor across the whole range. This stability allows for greater predictability when producing lenses that are capable of correcting peripheral vision while optimizing on-axis visual acuity.

A seventh embodiment of a set of lenses comprises at least one first lens having a refractive index between 1.50 and 1.60, a diopter label power of between 20 D and 27 D with a power shape factor between −1.0 and −2.4, and at least one second lens having a refractive index of between 1.40 and 1.50, a diopter label power between 24 D and 29 D and with a power shape factor that is between −0.3 and −0.8. The lenses within this set of lenses comprise sub-ranges of label power and shape factor that may be selected in order to optimize the structural features of the IOL to result in minimized off-axis astigmatism at 20 degrees and a maximized on-axis visual acuity to thereby improve peripheral vision.

The eighth embodiment of a set of lenses comprises at least one first lens having a refractive index between 1.40 and 1.50, a diopter label power of between 18 D and 22 D with a power shape factor between −1.1 and −2.0, and at least one second lens having a refractive index between 1.50 and 1.60, a diopter label power between 25 D and 35 D and with a power shape factor that is between −0.7 and −0.95. The lenses within this set of lenses comprise sub-ranges of label power and shape factor that may be selected in order to optimize the structural features of the IOL to result in minimized off-axis astigmatism at 20 degrees and a maximized on-axis visual acuity to thereby improve peripheral vision.

This disclosure uses the power shape factor ranges to further illustrate how lenses according to this disclosure can have ranges of structural values that ensure proper functioning and enhanced peripheral vision upon implantation in a patient.

It is further envisioned that any of the lenses disclosed herein may also correct for the average corneal spherical aberration as specified in ISO 11979-2 2014, Eye model #2.

### INDUSTRIAL APPLICATION

A method of designing a set of intraocular lenses for improved peripheral vision may be provided. The method may comprise providing an IOL having an optical power that reduces optical errors in an image produced at a peripheral retinal location of a patient's eye disposed at a distance from the fovea. An IOL may be provided for each increment of 0.25 D for the range 17 D to 35 D. As the label power of the lens increases from 17 D to 35 D, the power shape factor increases from −3.0 to −0.2. For the label powers corresponding to a shape factor below the transition point power, the IOL comprises a first lens body having a power shape factor less than or equal to minus one according to the embodiments discussed above. For the label powers corresponding to a shape factor greater than the transition point power, the IOL comprises a second lens body having a power shape factor greater than or equal to minus one according to the embodiments discussed above. Each IOL may have a blur parameter of less than 1.8 D.

Other methods of designing a set of lenses are envisioned within the scope of this disclosure.

An intraocular lens in accordance with this disclosure may also utilize complex surfaces, such as Zernike surfaces and toroidal surfaces, in addition to other variable, non-uniform surface structures for the anterior surface and posterior surface of a lens body. In one non-limiting embodiment of an IOL, the first surface has an anterior radius and exhibits an anterior power; a second surface has a posterior radius and exhibits a posterior power. At least one of the first surface or the second surface may be a complex surface. The intraocular lens exhibits a power shape factor, relative to a constant plano-convex power shape factor and corresponding to a diopter label power, wherein the power shape factor comprises a first power value, calculated with the anterior radius and the posterior radius, and a second power value, calculated with the anterior power and the posterior power. The first power value and the second power value may have equal or different values for a respective diopter label power. At least one of the first surface or the second surface has a refractive profile. The complex surfaces, therefore, may use variable anterior and posterior radius values and variable first power values. The complex surface further may also include a validated power shape factor having only the second power factor calculated with the anterior power and the posterior power. In the embodiments, at least one of the first surface or the second surface includes a toric surface. The power shape factor for the intraocular lens has a toric surface with an average of power meridians calculated for distinct portions of the intraocular lens surfaces. In additional embodiments, additional power may be added to the diopter label power, wherein the power shape factor remains equal to the second power value calculated with a base anterior power and a base posterior power.

In another embodiment of an intraocular lens according to this disclosure, the IOL has a first surface having an anterior radius and exhibiting an anterior power and a second surface having a posterior radius and exhibiting a posterior power. Optionally, at least one of the first surface or the second surface is a complex surface having multiple curvatures across the surface. Like other lenses described above, the intraocular lens exhibits a power shape factor, relative to a constant plano-convex power shape factor and corresponding to a label power measured in diopters. The power shape factor may have a first power value, calculated with the anterior radius and the posterior radius, and a second power value, calculated with the anterior power and the posterior power. The first power value and the second power value may have different values for a respective diopter label power. In non-limiting lenses of this disclosure, the complex surface may have variable anterior and posterior radius values and variable first power values. The complex surface may further include a validated power shape factor calculated with only the second power factor, which in turn, is calculated with the overall anterior surface power and the overall posterior surface power, in which overall powers account for the different radii of curvature. In some embodiments, at least one of the first surface or the second surface includes a toric surface (i.e., exhibiting a torus or toroidal shape across at least a portion of the surface). The power shape factor for the intraocular lens having a toric surface may be an average of power meridians calculated for distinct portions of the intraocular lens. In some cases, IOLs of this disclosure have additional power added to the label power, wherein the power shape factor remains equal to the second power value calculated with a base anterior power and a base posterior power.

In some situations, this disclosure describes relationships between the optical components in terms of position or in terms of operating parameters being comparable to each other. These descriptions are not limiting of the disclosure but are presented for example purposes only. In fact, when this disclosure uses numeric values for dimensions or ranges, all numeric values are understood to be “about” or “approximately equal,” and these phrases should be given the broadest plain meaning in the context of the technology. In some embodiments magnitudes of certain optical parameters may be “about” a certain value or “approximately equal” if the magnitudes differ from each other by an amount that is within a range selected from 0-5 percent of the larger value. Ranges in this disclosure are inclusive of endpoints unless otherwise specified or unless the context of the range dictates otherwise.

In addition to the background discussion above, this disclosure incorporates certain contextual information regarding example structures for IOLs, the optical effects of these structures on patient vision, and the environment in which IOLs are successfully used. As would be expected, numerous diagnostic steps occur before a physician prescribes an IOL for a patient. Measurements of a patient's eye may be made in a clinical setting, such as by an optometrist, ophthalmologist, or other medical or optical professional. The measurements may be made via manifest refraction, autorefraction, tomography, or a combination of these methods or other measurement methods. The optical aberrations of the patient's eye may also be determined. A determination of the visual range of the patient may also be determined. For example, the ability of the patient to focus on near objects (presbyopia) may be measured and determined. A range of add power for the ophthalmic lens may be determined.

The measurements of the patient's eye may be placed in an ophthalmic lens prescription, which includes features of at least one optic that is intended to address the optical aberrations of the patient's eye, as well as features that address the visual range for the patient (e.g., an amount of add power and number of focuses to be provided by the optic).

The ophthalmic lens prescription may be utilized to fabricate an optic for the ophthalmic lens. A refractive profile of the optic may be determined based on the ophthalmic lens prescription, to correct for the optical aberrations of the patient's eye. Such a refractive profile may be applied to the optic, whether on a surface including the diffractive profile or on an opposite optical surface. The diffractive profile may also be determined to provide for the desired distribution of add power for the optic.

The determination of one or more of a refractive or diffractive profile and the fabrication of the optic may be performed remotely from the optometrist, ophthalmologist, or other medical or optical professional that performed the measurements of a patient's eye, or may be performed in the same clinical facility of such an individual. If performed remotely, the fabricated optic may be delivered to an optometrist, ophthalmologist, or other medical or optical professional, for being provided to a patient. For an intraocular lens, the fabricated optic may be provided for implant into a patient's eye. The fabricated optic may be made according to the embodiments of this disclosure.

The fabricated optic may be a custom optic fabricated specifically for the patient's eye, or may be fabricated in a manufacturing assembly and then selected by an optometrist, ophthalmologist, or other medical or optical professional for supply to a patient, which may include implantation in the patient's eye.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, components, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed component essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

Various aspects of the subject matter described herein are set out in the following numbered examples, which may or may not be claimed:
1. An intraocular lens comprising:
   a lens body, wherein the lens body comprises:
   a refractive index between 1.40 and 1.50 inclusive, a diopter label power between 17 D and 23 D, inclusive, and a power shape factor that is less than or equal to −1.0 and greater than or equal to −2.5.
2. The intraocular lens according to Example 1, wherein the lens comprises a diopter label power that is between 18 D and 22 D, inclusive.
3. The intraocular lens according to Example 2, wherein the power shape factor is less than or equal to −1.1 and greater than or equal to −2.0.
4. The intraocular lens according to Example 1, wherein the lens comprises a central thickness that is between 0.62 mm and 1.0 mm.
5. The intraocular lens according to Example 2 or Example 3, wherein the lens comprises a central thickness that is between 0.70 mm and 0.90 mm.
6. The intraocular lens according to Example 1 or Example 4, further comprising an anterior haptic connected to the lens body, and wherein the lens comprises a vault height that is between 0.34 mm and 0.65 mm.
7. The intraocular lens according any one of Examples 2, 3 or 5, further comprising an anterior haptic connected to the lens body, and wherein the lens comprises a vault height that is between 0.40 mm and 0.60 mm.
8. The intraocular lens according to any one of Examples 1, 4 or 6, wherein the lens comprises a first surface having an anterior surface curvature greater than −0.077 mm⁻¹ and less than 0.00 mm⁻¹ and a second surface having a posterior surface curvature greater than −0.355 mm⁻¹ and less than −0.130 mm⁻¹.
9. The intraocular lens according to any one of Examples 2, 3, 5 or 7, wherein the lens comprises a first surface having an anterior surface curvature greater than −0.071 mm⁻¹ and less than 0.00 mm⁻¹ and a posterior surface curvature greater than −0.355 mm⁻¹ and less than −0.134 mm⁻¹.
10. An intraocular lens comprising:
   a lens body, wherein the lens body comprises:
   a refractive index between 1.50 and 1.60 inclusive, a diopter label power between 20 D and 28 D, inclusive, and a power shape factor that is less than or equal to −1.0 and greater than or equal to −3.
11. The intraocular lens according to Example 10, wherein the lens comprises a diopter label power that is between 20 D and 27 D.
12. The intraocular lens according to Example 11, wherein the power shape factor is less than or equal to −1.0 and greater than or equal to −2.4.
13. The intraocular lens according to Example 10, wherein the lens comprises a central thickness that is between 0.45 mm and 1.0 mm.
14. The intraocular lens according to Example 11 or Example 12, wherein the lens comprises a central thickness that is between 0.55 mm and 0.90 mm.
15. The intraocular lens according to Example 10 or Example 13, further comprising an anterior haptic connected to the lens body, and wherein the lens comprises a vault height that is between 0.30 mm and 0.65 mm.
16. The intraocular lens according to Example 11, Example 12 or Example 14, further comprising an anterior haptic connected to the lens body, and wherein the lens comprises a vault height that is between 0.35 mm and 0.60 mm.
17. The intraocular lens of Example 1 or Example 10, wherein the first surface of the lens comprises a concave surface and the second surface comprises a convex surface relative to an optical axis of the lens.
18. The intraocular lens of Example 17, wherein the concave surface comprises an anterior curvature radius of the concave surface that is larger than a posterior curvature radius of the convex surface.
19. The intraocular lens according to any one of Examples 10, 13 or 15, wherein the lens comprises a first surface having an anterior surface curvature greater than −0.022 mm⁻¹ and less than 0.00 mm⁻¹ and a second surface having a posterior surface curvature greater than −0.170 mm⁻¹ and less than −0.081 mm⁻¹.
20. The intraocular lens according to any one of Examples 11, 12, 14 or 16, wherein the lens comprises a first surface having an anterior surface curvature greater than −0.022 mm⁻¹ and less than 0.00 mm⁻¹ and a second surface having a posterior surface curvature greater than −0.170 mm⁻¹ and less than −0.084 mm⁻¹
21. An intraocular lens comprising:
   a lens body, wherein the lens body comprises:
   a refractive index between 1.40 and 1.50 inclusive, a diopter label power between 23 D and 30 D, inclusive, and a power shape factor that is less than or equal to −0.2 and greater than or equal to −1.
22. The intraocular lens according to Example 21, wherein the lens comprises a diopter label power that is between 24 D and 29 D, inclusive.
23. The intraocular lens according to Example 22, wherein the power shape factor is less than or equal to −0.3 and greater than or equal to −0.8.
24. The intraocular lens according to Example 21, wherein the lens comprises a central thickness that is between 0.62 mm and 1.0 mm.
25. The intraocular lens according to Example 22 or Example 23, wherein the lens comprises a central thickness that is between 0.70 mm and 0.90 mm.
26. The intraocular lens according to Example 21 or Example 24, further comprising an anterior haptic connected to the lens body, and wherein the lens comprises a vault height that is between 0.34 mm and 0.65 mm.
27. The intraocular lens according to any one of Examples 22, 23 or 25, further comprising an anterior haptic connected to the lens body, and wherein the lens comprises a vault height that is between 0.40 mm and 0.60 mm.
28. The intraocular lens according to any one of Examples 21, 24 or 26, wherein the lens comprises a first surface having an anterior surface curvature greater than 0 mm⁻¹ and less than 0.120 mm⁻¹ and a second surface having a posterior surface curvature greater than −0.367 mm⁻¹ and less than −0.130 mm⁻¹.
29. The intraocular lens according to any one of Examples 22, 23, 25 or 27, wherein the lens comprises a first surface having an anterior surface curvature greater than 0 mm⁻¹ and less than 0.12 mm⁻¹ and a second surface having a posterior surface curvature greater than −0.36 mm⁻¹ and less than −0.130 mm⁻¹.
30. An intraocular lens comprising:
   a lens body, wherein the lens body comprises:
      a refractive index between 1.50 and 1.60 inclusive, a diopter label power between 23 D and 35 D, inclusive, and a power shape factor that is less than or equal to −0.2 and greater than equal to −1.
31. The intraocular lens of Example 30, wherein the diopter label power is between 25 D and 35 D, inclusive.
32. The intraocular lens of Example 31, wherein the power shape factor is less than or equal to −0.40 and greater than or equal to −0.95.
33. The intraocular lens according to Example 30, wherein the lens comprises a central thickness that is between 0.45 mm and 1.0 mm.
34. The intraocular lens according to Example 31 or Example 32, wherein the lens comprises a central thickness that is between 0.55 mm and 0.90 mm.
35. The intraocular lens according to Example 30 or Example 33, further comprising an anterior haptic connected to the lens body, and wherein the lens comprises a vault height that is between 0.30 mm and 0.65 mm.
36. The intraocular lens according to any of Examples 31, 32 or 34, further comprising an anterior haptic connected to the lens body, and wherein the lens comprises a vault height that is between 0.35 mm and 0.60 mm.
37. The intraocular lens according to any one of Examples 30, 33 or 35, wherein the lens comprises a first surface having an anterior surface curvature greater than 0.00 mm⁻¹ and less than 0.082 mm⁻¹ and a second surface having a posterior surface curvature greater than −0.179 mm⁻¹ and less than −0.081 mm⁻¹.
38. The intraocular lens according to any one of Examples 31, 32, 34 or 36, wherein the lens comprises a first surface having an anterior surface curvature greater than 0.01 mm⁻¹ and less than 0.082 mm⁻¹ and a second surface having a posterior surface curvature greater than −0.179 mm⁻¹ and less than −0.081 mm⁻¹
39. The intraocular lens of any of Examples 1, 10, 21 or 30, wherein the power shape factor is calculated using paraxially defined curvature radii of the first surface and the second surface.
40. The intraocular lens of any of Examples 1, 10, 21 or 30, wherein the power shape factor is calculated according to the formula

$$\frac{Power ant - Power post}{Power ant + Power post} ,$$

wherein Powerₐₙₜ is the anterior power, and Powerₚₒₛₜ is the posterior power of the lens body.
41. The intraocular lens of any of Examples 1, 10, 21 or 30, wherein the power shape factor is calculated by ray tracing techniques applied to the intraocular lens.
42. The intraocular lens of any preceding Example 41, wherein the ray tracing techniques comprise utilizing a specific aperture size.
43. The intraocular lens of any preceding Example 41, wherein the ray tracing techniques comprise utilizing a specific aperture size, a best focus position and spherical aberration effects on the power shape factor.
44. The intraocular lens according to any of Examples 1 to 43, having an MTF value of at least 0.7 at a spatial frequency of 50 cycles/mm for a 5 mm pupil in green light.
45. The intraocular lens according to any of Examples 1 to 44, wherein the intraocular lens corrects for the average corneal spherical aberration as specified in ISO 11979-2 2014, Eye model #2.
46. The intraocular lens according to any of Examples 1 to 45, wherein the lens comprises an anterior surface that is aspheric, or wherein the lens comprises a posterior surface that is aspheric, or wherein the lens comprises a posterior surface and an anterior surface that are aspheric.
47. A set of intraocular lenses that improve peripheral vision, the set comprising:
   at least one first lens according to any of Examples 1 to 20; and
   at least one second lens according to any of Examples 21 to 38.
48. The set of intraocular lenses according to Example 47, wherein the at least one first lens comprises a series of lenses wherein the diopter label power of each lens of the series of lenses differs by at least 0.25 diopters.
49. The set of intraocular lenses according to Example 47, wherein the at least one second lens comprises a series of lenses wherein the diopter label power of each lens of the series of lenses differs by at least 0.25 diopters.
50. A method of designing a set of intraocular lenses for improved peripheral vision wherein as the label power of the lens increases from 17 D to 35 D, the power shape factor increases from −2.5 to −0.2, the method comprising:
for each label power increment of at least 0.25 diopters, providing an IOL having an optical power that reduces optical errors in an image produced at a peripheral retinal location of a patient's eye disposed at a distance from the fovea,
wherein for label powers corresponding to a shape factor less than or equal to −1, the IOL comprises a lens according to any of Examples 1 to 20,
wherein for label powers corresponding to a shape factor greater than or equal to −1, the IOL comprises a lens according to any of Examples 21 to 38,
wherein the blur parameter of each IOL is less than 1.8 diopters, wherein the blur parameter is calculated according to: Blur Parameter=\sqrt{Sphere²+Cylinder²}.

What is claimed is:

1-20. (canceled)

21. An intraocular lens comprising:
a lens body, wherein the lens body comprises:
a refractive index between 1.40 and 1.50 inclusive, a diopter label power between 23 D and 30 D, inclusive, and a power shape factor that is less than or equal to −0.2 and greater than or equal to −1.

22. The intraocular lens according to claim 21, wherein the lens comprises a diopter label power that is between 24 D and 29 D, inclusive.

23. The intraocular lens according to claim 22, wherein the power shape factor is less than or equal to −0.3 and greater than or equal to −0.8.

24. The intraocular lens according to claim 21, wherein the lens comprises a central thickness that is between 0.62 mm and 1.0 mm.

25. The intraocular lens according to claim 22, wherein the lens comprises a central thickness that is between 0.70 mm and 0.90 mm.

26. The intraocular lens according to claim 21, further comprising an anterior haptic connected to the lens body, and wherein the lens comprises a vault height that is between 0.34 mm and 0.65 mm.

27. The intraocular lens according to claim 22, further comprising an anterior haptic connected to the lens body, and wherein the lens comprises a vault height that is between 0.40 mm and 0.60 mm.

28. The intraocular lens according to claim 21, wherein the lens comprises a first surface having an anterior surface curvature greater than 0 mm⁻¹ and less than 0.120 mm⁻¹ and a second surface having a posterior surface curvature greater than −0.367 mm⁻¹ and less than −0.130 mm⁻¹.

29. The intraocular lens according to claim 22, wherein the lens comprises a first surface having an anterior surface curvature greater than 0 mm⁻¹ and less than 0.12 mm⁻¹ and a second surface having a posterior surface curvature greater than −0.36 mm⁻¹ and less than −0.130 mm⁻¹.

30. An intraocular lens comprising:
a lens body, wherein the lens body comprises:
a refractive index between 1.50 and 1.60 inclusive, a diopter label power between 23 D and 35 D, inclusive, and a power shape factor that is less than or equal to −0.2 and greater than equal to −1.

31. The intraocular lens of claim 30, wherein the diopter label power is between 25 D and 35 D, inclusive.

32. The intraocular lens of claim 31, wherein the power shape factor is less than or equal to −0.40 and greater than or equal to −0.95.

33. The intraocular lens according to claim 30, wherein the lens comprises a central thickness that is between 0.45 mm and 1.0 mm.

34. The intraocular lens according to claim 31, wherein the lens comprises a central thickness that is between 0.55 mm and 0.90 mm.

35. The intraocular lens according to claim 30, further comprising an anterior haptic connected to the lens body, and wherein the lens comprises a vault height that is between 0.30 mm and 0.65 mm.

36. The intraocular lens according to claim 31, further comprising an anterior haptic connected to the lens body, and wherein the lens comprises a vault height that is between 0.35 mm and 0.60 mm.

37. The intraocular lens according to claim 30, wherein the lens comprises a first surface having an anterior surface curvature greater than 0.00 mm⁻¹ and less than 0.082 mm⁻¹ and a second surface having a posterior surface curvature greater than −0.179 mm⁻¹ and less than −0.081 mm⁻¹.

38. The intraocular lens according to claim 31, wherein the lens comprises a first surface having an anterior surface curvature greater than 0.01 mm⁻¹ and less than 0.082 mm⁻¹ and a second surface having a posterior surface curvature greater than −0.179 mm⁻¹ and less than −0.081 mm⁻¹.

39. The intraocular lens of any of claim 1, 10, 21 or 30, wherein the power shape factor is calculated using paraxially defined curvature radii of the first surface and the second surface.

40. The intraocular lens of any of claim 1, 10, 21 or 30, wherein the power shape factor is calculated according to the formula
$$\frac{Power ant - Power post}{Power ant + Power post} ,$$
wherein Powerₐₙₜ is the anterior power, and Powerₚₒₛₜ is the posterior power of the lens body.

41. The intraocular lens of any of claim 1, 10, 21 or 30, wherein the power shape factor is calculated by ray tracing techniques applied to the intraocular lens.

42. The intraocular lens of claim 41, wherein the ray tracing techniques comprise utilizing a specific aperture size.

43. The intraocular lens of claim 41, wherein the ray tracing techniques comprise utilizing a specific aperture size, a best focus position and spherical aberration effects on the power shape factor.

44. The intraocular lens according to any of claims 1 to 43, having an MTF value of at least 0.7 at a spatial frequency of 50 cycles/mm for a 5 mm pupil in green light.

45. The intraocular lens according to any of claims 1 to 44, wherein the intraocular lens corrects for the average corneal spherical aberration as specified in ISO 11979-2 2014, Eye model #2.

46. The intraocular lens according to any of claims 1 to 45, wherein the lens comprises an anterior surface that is aspheric, or wherein the lens comprises a posterior surface that is aspheric, or wherein the lens comprises a posterior surface and an anterior surface that are aspheric.

47-50. (canceled)
